(19) United States
(12) Patent Application Publication
(10) Pub. No.: US 2003/0032149 A1
(43) Pub. Date: Feb. 13, 2003

(54) Synthesis of 2-oxoglutaramate from L-glutamine

(76) Inventor: James J. Lalonde, Boston, MA (US)

Correspondence Address: PPG INDUSTRIES, INC., Intellectual Property Department, One PPG Place, Pittsburgh, PA, 15272, US

(21) Appl. No.: 09/928,810

(22) Filed: Aug. 13, 2001

(51) Int. Cl. C12P 13/04; C12N 1/20

(52) U.S. Cl. 435/106; 435/252.1

(57) ABSTRACT

A fermentation method for the production of 2-oxoglutaramic acid including the step of incubating L-glutamine in the presence of bacteria of the genera Providencia and Proteus, including Providencia sp. PCM-1270 and PCM-1298 and Proteus mirabilis PCM-1353. The incubation typically is conducted in an aqueous slurry of solid L-glutamine, a buffer, catalase and the bacteria cells. Typically, a wet cell pellet mass of cells of at least 1% by weight of the incubation mixture is included in the L-glutamine-containing incubation mixture. A bacteria culture containing the bacteria and L-glutamine is provided along with a bacteria culture containing the bacteria and 2-oxoglutaramic acid, the product of the above-described method.

### BACKGROUND OF THE INVENTION

1. Field of the Invention

The present invention is directed to a method for producing 2-oxoglutaramate from L-glutamine and the product of that method.

2. Description of the Related Art

2-oxoglutaramate (also known as 2-hydroxy-5-oxoproline and α-ketoglutaramic acid) is synthesized and metabolized in plants by the sequential action of transaminase and hydrolase enzymes. Similar transaminase and hydrolase enzymes and 2-oxoglutaramate have been identified in animal livers and kidneys. These enzymes and 2-oxoglutaramate were partially characterized in Meister, A, “Enzymatic Preparation of a-Keto Acids,” J. Biochem. 197:304 (1952). The compound 2-oxoglutaramate has substantial agricultural uses as a plant growth regulator. However, economical and/or safe methods for the production of 2-oxoglutaramate do not exist. Meister, A, “Preparation and Enzymatic Reactions of the Keto Analogues of Asparagine and Glutamine J. Biol. Chem.,” 20, 571 (1953), describes the conversion of L-glutamine to 2-oxoglutaramate with rattlesnake (Crotalus adamanteus) and other snake venoms. While effective, use of snake venom for preparative conversion of L-glutamine to 2-oxoglutaramate is commercially impracticable. Oxidation of alpha amino acids by non-enzymatic means requires the use of highly reactive, inorganic oxidants such as Fremy's salt (A. Garcian-Raso, P. M. Deyà, and J. M. Saa, J. Org. Chem. 1986, 51, 4285-4287).

Bacteria of the Genera Proteus, Providencia and Morganella are known to deaminate phenylalanine to phenylpyruvic acid. Members of these genera also have the ability to convert amino acids other than phenylalanine to their corresponding α-keto acid. U.S. Pat. No. 5,728,555 discloses a recombinant L-aminodeaminase (Lad) gene derived from Proteus myxofaciens purportedly exhibiting the ability to convert small amounts of L-glutamine to another product. However, there is no indication that P. myxofaciens, the source of the Lad gene described in that patent, can convert L-glutamine to 2-oxoglutaramate and not subsequently convert the product to a secondary product. U.S. Pat. Nos. 4,614,714, 4,783,404 and 5,801,035 disclose L-amino acid oxidases of Streptomyces, Cryptococcus and Trichoderma species. None of the L-amino acid oxidases of the above-referenced patents are known to have the ability to convert L-glutamine to 2-oxoglutaramate in preparative quantities.

Each of the above-described organisms converts a different panel of amino acids to their respective α-keto acids. In many instances, even if those organisms can convert L-amino acids to their corresponding α-keto acid, the α-keto acid is an intermediate that is rapidly converted to secondary products. It is not known if those organisms are suitable for preparative production of 2-oxoglutaramate from L-glutamine.

### SUMMARY

It has now been found that members of the Providencia and Proteus genera have the capacity to convert L-glutamine to 2-oxoglutaramate in bulk, without subsequent conversions of 2-oxoglutaramate to secondary products. Provided herein is a biocatalytic method for converting L-glutamine to 2-oxoglutaramate including the step of contacting L-glutamine with bacteria of the genera Providencia and Proteus, or an active biocatalyst derived therefrom. Also provided are reaction mixtures containing Providencia or Proteus bacteria, or active biocatalysts derived from the bacteria, the reaction mixtures containing preparative amounts of L-glutamine and/or 2-oxoglutaramate.

The method for producing 2-oxoglutaramate includes incubating Providencia or Proteus bacteria or an active biocatalyst derived therefrom in an incubation solution comprising at least about 25 mM (millimolar) L-glutamine. Specific Providencia and Proteus strains include, without limitation, Providencia sp. PCM-1298, ATCC Deposit No. PTA-3563 Providencia sp. PCM-1270, ATCC Deposit No. PTA-3563, and Proteus mirabilis PCM-1353, ATCC Deposit No. PTA-3562. The L-glutamine typically is added to the incubation solution prior to addition of the bacteria. Additionally, or alternatively, L-glutamine may be added to a culture already containing bacteria. The L-glutamine may be added as a single aliquot, or continuously over a period of time, for continuous-feed processes, either in two or more aliquots or as a steady trickle. The incubation solution may be an aqueous slurry comprising solid L-glutamine.

Also provided is a reaction mixture including Providencia or Proteus bacteria, or an active biocatalyst derived therefrom, and at least about 25 mM L-glutamine, representing a culture for preparative production of 2-oxoglutaramate. A composition also is provided including Providencia or Proteus bacteria, or an active biocatalyst derived therefrom, and at least about 25 mM 2-oxoglutaramate, representing a culture that is an end product of the above-described method.

### DETAILED DESCRIPTION OF THE PREFERRED EMBODIMENTS

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within these ranges. Also, the disclosure of these ranges is intended as a continuous range including every value between the minimum and maximum values.

Described herein is a method for producing 2-oxoglutaramate, a chemical compound with value as a plant growth regulator. Current manufacturing methods for producing 2-oxoglutaramate are not commercially practicable. The method of the present invention is a process by which L-glutamine is incubated in the presence of bacteria of the Providencia genus, such as Providencia sp., and including Providencia strains PCM-1270, ATCC Deposit No. PTA-3564, and PCM-1298, ATCC Deposit No. PTA-3563, and/or bacteria of the Proteus genus, for instance Proteus mirabilis, such as strain PCM-1353, ATCC Deposit No. PTA-3562, each of which were deposited on Aug. 23, 2001 at the American Type Culture Collection (ATCC), 10801 University Boulevard, Manassas, Va. 20110-2209, U.S.A. The designation “PCM-XXXX” refers to accession numbers of the Polish Collection of Microorganisms, Institute of Immunology and Experimental Therapy, Wroclaw, Poland.

The method comprises the step of culturing the Providencia or Proteus bacteria in the presence of high (preparative) concentrations of L-glutamine. By “high concentrations,” it is meant concentrations of convertible L-glutamine higher than those concentrations of convertible L-glutamine found in standard culture media for Providencia bacteria. The choice of a suitable culture medium may vary, and may include, without limitation, the following standard culture media for culturing bacteria of the Providencia genus:

(I) Luria-Bertani (LB) medium

10 g/L tryptone

5 g/L yeast extract, and

10 g/L NaCl adjusted to pH 7.4; or

(II) Szwajcer et al. Providencia medium

10 g/L bactopeptone

2 g/L casein hydrolysate

2 g/L yeast extract

6 g/L NaCl or 10 mm CaCl₂

Additional suitable culture media for growth of Providencia or Proteus bacteria, as well as modifications to the suitable culture media described above, will be known to those skilled in the art.

While the culture media described above may be best suited to grow large, preparative quantities of the bacteria described herein, it may not be optimally suited for use in the commercial preparation of 2-oxoglutaramate due to impurities present in the media. To produce 2-oxoglutaramate in as pure a form as possible, the culture medium should include the least amount of ingredients necessary for sustainable (over many hours, typically from one to 48 hours) conversion of L-glutamine to 2-oxoglutaramate. Therefore, the bacteria cells are typically grown under optimal growth conditions in standard culture media, collected from culture by centrifugation or filtration as a wet cell pellet and resuspended in an incubation solution containing L-glutamine and a minimum of additional ingredients. For instance, and without limitation, a suitable incubation solution is 50 mm Tris (Tris-(hydroxymethyl) aminomethane) pH 8, 100 mM L-glutamine and catalase (10 ml/L of T100, Genencor International, Inc.). The buffer and catalase may be omitted, depending on the culture conditions and the biocatalyst used. From 10 g/L to 500 g/L of wet cell paste may be added to the incubation solution and the culture is agitated and oxygenated for an amount of time suitable to convert L-glutamine to 2-oxoglutaramate, typically from 1 to 24 hours, more typically from 5-15 hours. The relative amounts of ingredients, the choice of ingredients and length of incubation time will vary, depending on culture conditions and form of the biocatalyst and may be optimized for overall yield of 2-oxoglutaramate product.

As used herein, the wet cell mass, paste or pellet (“wet cell pellet”) is prepared by removal of culture medium from a culture of bacteria cells. Culture medium can be removed, for example and without limitation, by centrifugation or filtration. Centrifugation may be performed in a variety of manners, as are known in the art. Centrifugation conditions may vary, so long as amino acid oxidase activity of the cultured cells is retained, and preferably optimized. In preparing a wet cell mass, the bacteria cells typically remain viable. Filtration also may be used to prepare the cell paste. Suitable filtration units include, without limitation, 0.4 micron nylon filters or filter units, such as those commercially available from Millipore Corporation of Bedford, Mass.

Different centrifugation and filtration methods theoretically will yield slightly different cell densities in a wet cell pellet. As used herein, for standardization, a “wet cell pellet” of bacteria refers to a mass of cells prepared by ultracentrifugation according to the method of Example 1, below; by ultracentrifugation at 10,000 g for 10 minutes.

To remove impurities from the cell paste, the paste may be washed one or more times with a suitable wash solution. The wash solution may be water or a buffer solution, and can be the same incubation solution in which the L-glutamine is converted.

The culture medium or incubation solution for preparation of 2-oxoglutaramate includes L-glutamine, typically at least about 25 mM (about 3.2 g/L), preferably at least about 100 mM and most preferably at least about 250 mM. In another embodiment, the culture is a slurry of L-glutamine. In a slurry, the incubation solution contains solid L-glutamine, so that a substantially saturated concentration of L-glutamine is maintained. A typical range of solid L-glutamine in a slurry is from a trace of L-glutamine solid (>0 g/L) to at least about 250 g/L of L-glutamine. For example, as shown herein a slurry containing about 25% w/v (250 g/L) L-glutamine efficiently converts L-glutamine to 2-oxoglutaramate. In a slurry, the maximum amount of L-glutamine that can be present in the incubation solution, which can be in excess of 250 g/L, is limited by the maximum amount of solid L-glutamine that can be present while retaining adequate conversion. This maximum amount may vary, depending upon the oxygenation and agitation means used in any given instance. Because 2-oxoglutaramate is much more soluble than L-glutamine in aqueous solutions under typical bioconversion conditions (about 15 times more soluble), the solid L-glutamine is converted into dissolved 2-oxoglutaramate. Therefore, in a typical reaction, once the reaction is complete, no solid will be left in the incubation solution. However, if enough starting material is used, solid 2-oxoglutaramate will form once a saturating amount of that product is produced. In such a case, the solid 2-oxoglutaramate may be purified by filtration and may be washed with ethanol or resuspended in water and precipitated with cold ethanol.

As an alternative to formation of a slurry, L-glutamine may be fed into the culture over a pre-determined period of time as a continuous stream or by addition of multiple aliquots of L-glutamine to the culture. The L-glutamine may be fed into the culture by standard methods, for instance, as a solution by a peristaltic pump or by a drip. The drawback of this method is that the addition of solubilized L-glutamine increases the volume of the incubation solution. This embodiment may be implemented under conditions of continued logarithmic growth of the bacteria culture, where the incubation solution is L-glutamine-supplemented bacteria growth medium and the feed L-glutamine is dissolved in bacteria growth medium. In this embodiment, 2-oxoglutaramate-containing medium may be periodically removed. For instance, 500 ml of L-glutamine-containing medium may be added to a 1 L culture over a period of one hour, during which or after which 500 ml may be removed. Suitable continuous feed fermentation processes and apparatus are known.

As used herein, “near-saturation” is an approximation of a concentration of L-glutamine approaching, but not including 100% saturation (“saturated”). Practically, “near-saturation” means from about 75% up to a 100% saturated solution, preferably at least about 90% saturated. By having a culture near-saturation, no solid L-glutamine is present, but a maximum practicable amount of dissolved L-glutamine is made available for conversion to 2-oxoglutaramate.

Buffer(s) useful in the incubation solution include, without limitation, phosphate buffers, TRIS, PIPES (piperazine-N,N′-bis (ethanesulfonic acid), Bis-Tris (bis(2-hydroxyethyl)imino-tris (hydroxymethyl) methane), HEPES (N-2-hydroxyethylpiperazine-N-2-ethane sulfonic acid) and MOPS (3-(N-morpholino) propane sulfonic acid), or mixtures thereof commercially available from Research Organics, Inc. of Cleveland, Ohio among others. Useful buffer concentrations will vary depending upon the choice of buffer(s), and the reaction conditions. In reference to Tris-Cl buffers, useful ranges typically vary from 10 mM to 250 mM, more typically from 25 mM to 150 mM and most typically from 50 mM to 100 mM. Suitable pH ranges will vary and may be optimized empirically. Typically, the pH will range from 6.5 to 8.5, more typically from 7.0 to 8.0. In any case, these buffer concentrations and pH ranges may be optimized to maximize 2-oxoglutaramate production for any culture condition.

It also should be noted that L-glutamine and 2-oxoglutaramate have buffering capacity. As such, no additional buffer may be needed to buffer the incubation solution. Further, in large-scale applications, the pH of the incubation solution can be regulated by a control system that includes a pH meter and a means for automatically depositing a simple acid (for instance HCl) or a simple base (for instance NaOH) into the incubation solution to regulate the pH of the incubation solution. This may be the most favorable method to regulate pH in that simple acids and bases are less expensive than buffers, and they form simple salts which may not interfere with the purification process as would more complex buffers.

In aqueous solution, 2-oxoglutaramate rapidly decarboxylates in the presence of hydrogen peroxide. For this reason, when hydrogen peroxide is present or expected as a co-product of the conversion process, a hydrogen peroxide-decomposing agent such as catalase or manganese dioxide may be added to the culture. Catalase is commercially available from Genencor International, Inc. of Rochester, N.Y. The amount of catalase added to-the culture will depend on the culture conditions, typically, the amount of L-glutamine to be converted. The amount of catalase added also may depend upon the source of the catalase enzyme preparation. For example, 10 ml to 50 ml of Genencor's T100 catalase preparation was used in the Examples, below (T100 catalase contains about 1000 Baker Units (BU) of enzyme per ml). As little as 1 ml/L (1,000 BU/L), and as much as 100 ml/L (100,000 BU/L) of the T100 catalase, or its functional equivalent, may be added to the reaction mixture. Higher or lower concentrations of catalase may be used, depending on reaction conditions. Other hydrogen peroxide-decomposing agents may be used, such as, without limitation, enzymes other than catalase or inorganic compounds such as manganese dioxide, platinum or iron oxide.

It should be noted that no hydrogen peroxide, or only slight traces thereof, was detected whenever the experiments described in the Examples below were run without catalase. This could mean a variety of things, without limitation: 1) that the enzyme does not produce hydrogen peroxide, 2) that the microbe also produces a catalase, or 3) that the peroxide reacts with components in the incubation solution other than 2-oxoglutaramate. In view of this, catalase is not necessarily required in the reaction mixture, but may be used whenever hydrogen peroxide is either present or contributes to further conversion of the 2-oxoglutaramate product.

The culture or reaction mixture is agitated and oxygenated as necessary to provide optimal yields of 2-oxoglutaramate. The culture may be agitated by any means known in the art, including without limitation shaking and stirring. The oxygenation may be accomplished by agitation, by bubbling air or oxygen through the culture or by any other suitable means. For example, as described below, air may be bubbled through the reaction mixture through a sintered glass sparger. Oxygenation of slurries also may be conducted in any manner known in the art, such as by agitation, stirring or bubbling or combinations thereof.

Production of 2-oxoglutaramate may be monitored by HPLC or by any method capable of determining production of 2-oxoglutaramate. A typical completed reaction will contain at least about 20 mM to about 25 mM 2-oxoglutaramate (corresponding to the aforementioned at least about 25 mM L-glutamine as starting material and assuming 80% to 100% conversion).

Besides whole cell biocatalytic conversion of L-glutamine to 2-oxoglutaramate, as described above, 2-oxoglutaramate may be produced from L-glutamine using active biocatalyst derived from the above-described cells or cell cultures. By “active”, it is meant possessing the ability to convert L-glutamine to 2-oxoglutaramate. By “active biocatalyst” it is meant any cell; cell product; cell or cell culture extract, fraction or product or chemically, physically or biologically modified derivatives thereof that possesses the ability to convert L-glutamine to 2-oxoglutaramate. Active biocatalysts include: cells; cell homogenates; disrupted cells; whole cell extracts; cell fractions isolated by any physical, chemical or biological means; protein fractions; cell culture supernatant and chemically modified active derivatives thereof. Active biocatalysts may be immobilized by attachment to a surface, including without limitation beads, culture plates and porous structures including foamed polymeric materials and ceramics, according to methods known in the art.

Immobilized active biocatalysts may be particularly useful in commercial preparation of 2-oxoglutaramate due to their capacity for re-use. In many cases, the L-glutamine can be fed to the immobilized active biocatalyst in water, thereby removing additional contaminates from the incubation solution, resulting in a product that can be purified very easily. For instance, the biocatalyst can be immobilized in a column and the L-glutamine is added to the column and is converted to 2-oxoglutaramate by passage through the column.

Once a desired amount of 2-oxoglutaramate is produced according to the whole cell biocatalytic methods described herein, the conversion reaction may be stopped. One method for stopping the reaction is by removing the bacteria cells by filtration or by centrifugation. The filtrate or supernatant can be frozen at this stage, typically at temperatures less than −20° C., or may be otherwise stabilized and/or preserved. Other methods for the stabilization and/or preservation of the product may be by pH control of the solution or by removal of water to form a lyophilized, low-water content or substantially water-free formulation. These stabilization and/or purification methods may be used for any 2-oxoglutaramate solution prepared by any method described herein.

For many uses for the 2-oxoglutaramate, such as agricultural uses, the reaction mixture may be used as-is or with the bacteria killed or disrupted, with nothing further done to purify and/or concentrate the product. Ultrasound, freezing and thawing or mechanical methods are, without limitation, methods that may be used to disrupt the bacteria cells. The cells may be killed by irradiation with, for example, gamma or ultraviolet irradiation.

If desired, the 2-oxoglutaramate may be purified. As described above, in a whole cell biocatalytic method, the reaction may be stopped and the bacteria may be removed from the incubation solution by mechanical means, such as by filtration or ultracentrifugation. In the case of filtration, the reaction mixture is filtered through a suitable filter, such as, without limitation a 0.2 or 0.4 micron nylon membrane (e.g., Millipore filters). Other suitable filters and filtration devices are broadly available. The filters may be washed with water or buffer, as desired. Centrifugation may be performed under standard conditions, for instance by ultracentrifugation at 10,000 g for 10 minutes.

2-oxoglutaramate can be purified by standard methods, for instance by chromatographic methods, by pH precipitation, by solvent precipitation or by precipitation of metal salts of the product. Metal salts include, without limitation, calcium, barium or zinc salts. If the 2-oxoglutaramate is produced by passage over an immobilized biocatalyst with a minimum of additional ingredients, there may be such low levels of additional ingredients in the incubation solution that the solution may be evaporated, leaving substantially pure, solid 2-oxoglutaramate product. In a production facility, the evaporation process might immediately follow the conversion process, thereby reducing the opportunity for further conversion to undesired products.

One suitable chromatographic method for purification of the product is ion exchange chromatography; for example and without limitation by ion exchange chromatography over a Dowex HCR-W2 ion exchange resin, acid form. The eluate from the resin containing the 2-oxoglutaramate product (typically a greater than 80% yield of 2-oxoglutaramate) may be concentrated by standard methods including evaporative techniques, distillation, extractions, precipitations and the like. The reaction product may be converted to its barium salt by adjusting the pH of the column eluant to about 5.0 with saturated barium hydroxide. Calcium hydroxide, or other metal salts, also maybe used in a similar fashion to convert the reaction product to its calcium salt, or other metal salt. The eluate may be concentrated by standard evaporative techniques and may be precipitated with cold ethanol.

### EXAMPLES

### Example 1

### Cell Culture

Microbes Providencia sp. PCM-1298, PCM-1270, and Proteus mirabilis PCM-1353 were obtained from the Polish Collection of Microorganisms, Wroclaw, Poland. Freeze dried samples were revived in liquid culture using a media consisting of bactopeptone (1%), casein hydrolysate (0.2%), yeast extract (0.2%) and NaCl (0.6%), pH 7.2. Szwajcer E. et al., Enzyme Microbiol. Tech., 1982, 409-413). Alternatively, cells were revived in solid culture using LB agar media. Liquid culture media (40 ml) was inoculated with a few milligrams of freeze-dried cells in 250 ml shake flasks. The flasks were agitated at 200 rpm in an incubator at 30° C. for 20 hours. The cells were collected by ultracentrifugation (10,000 g for 10 minutes) to remove the culture media and the pellet was washed twice by resuspension in 10 mM Tris, pH 7.0 buffer. A 1 g pellet (wet cell weight) was resuspended in 10 ml of the same wash buffer and used in subsequent biotransformations. Dry cell weight: Drying a 1 g wet cell pellet for 2 hours at 100° C. gave approximately 0.3 g of dry cell mass.

Alternatively, the cells can be recovered by filtration using a 0.4 micron membrane (such as a nylon membrane) or can be immobilized using standard methods (e.g. the calcium alginate method described by Szwajcer et al).

### Example 2

### Biocatalytic Deamination of Glutamine

Cell cultures were examined for their ability to catalyze the conversion of L-glutamine by adding 4 ml of the cell suspension of Example 1 to a 10 ml solution containing 50 mM Tris, pH 8.0; 1% w/v L-glutamine and 100 μl of catalase (T100, Genencor International). After 18 hours, conversion of L-glutamine and production of 2-oxoglutaramate was indicated by HPLC analysis. In the case of the Proteus mirabilis PCM-1353 strain, however, some further digestion of the 2 oxo-acid was indicated by the appearance of secondary products.

HPLC was performed under the following conditions:

1. Column: 15 cm, C18, 100 Å reverse phase Microsorb MV (Varian, Inc. of Walnut Creek, Calif.).

2. Mobile Phase: 0.1% aqueous solution of trifluoroacetic acid, 1 ml/min.

3. Detection: 220 nm

Retention times of reactant, product standard and byproduct standards (minutes): L-glutamine (1.7), 2-oxoglutaramate (2.2), succinamic acid (2.3), glutamic acid (1.9).

A calibration standard of the 2 oxo-acid was prepared according to the method of Meister using purified L-amino acid oxidase (Worthington Biochemical Corporation of Lakewood, N.J.). The product was purified by ion exchange chromatography and isolated as the barium salt. The ¹³C NMR spectrum for this compound was consistent with the predominant cyclized hydroxy proglutamate tautomer. Chemical shifts (ppm): 30.0, 34.1, 89.5, 178.0 and 182.0.

### Example 3

### Biocatalytic Deamination of Glutamine

L-glutamine (1 g) was dissolved in 20 ml of pH 7.0, 50 mM Tris buffer and the pH was readjusted to 7.0 with 1N NaOH. Catalase (200 μl, T100, Genencor International) was then added. To this solution was added 1 g of wet paste of either Providencia sp. PCM-1298 or PCM-1270. The solution was gently aerated with oxygen gas using a sintered glass sparger. The reaction was monitored by periodically removing aliquots and analyzing the aliquots by HPLC for the conversion of L-glutamine to 2-oxoglutaramate. Once the conversion of L-glutamine was complete, the reaction was stopped by ultracentrifugation (10,000 g for 10 minutes). Under these conditions, the yield of 2-oxoglutaramate was >80% as measured by HPLC.

### Example 4

### Preparative Deamination of L-Glutamine

L-Glutamine (50 g) was dissolved in 1000 ml of pH 8.0, 50 mM Tris buffer and the pH was readjusted to 8.0. Catalase (50 ml, T100, Genencor International) was then added. To this solution was added 25 g of wet cell paste of Providencia sp. PCM-1298. The solution was agitated in a 2-liter shake flask at 250 rpm in an orbital shaking incubator. The reaction was monitored periodically by HPLC by removing aliquots and analyzing for the conversion of L-glutamine to 2-oxoglutaramate. After 10 hours, the reaction was stopped by removal of the cell mass by ultracentrifugation (10,000 g for 10 minutes). The yield of 2-oxoglutaramate was approximately 85% by HPLC. The supernatant was frozen at −20° C. until ready for purification.

The supernatant was purified by ion exchange chromatography over 500 ml of Dowex HCR-W2 ion exchange resin. In this process unreacted L-glutamine and other contaminants are bound to the resin and pure 2-oxoglutaramate is eluted.

The resin was converted to the acid form by incubation with two volumes of 2M HCl. The column was then washed with distilled water until the pH of the eluate was over 4. The aqueous product solution was added to the column, followed by distilled water at a flow rate of 5 ml/min. The pH of the eluate was monitored and when the pH dropped to 2, 200 ml fractions were collected and analyzed by HPLC for the presence of 2-oxoglutaramate. Fractions containing 2-oxoglutaramate were pooled and the pH of the pooled fractions was adjusted to 7 with 2N NaOH. The eluate was treated with activated charcoal and filtered through a 0.2 micron sterile filter. The concentration of 2-oxoglutaramate at this point was 3.75%, indicating an overall yield of 75%.

### Example 5

### Preparative Deamination of L-Glutamine Slurry

L-Glutamine (2 g) was suspended in 8 ml of pH 8.0, 50 mM Tris buffer and the pH readjusted to 8.0 with 1 N NaOH. Catalase (200 μl, T100, Genencor International) was then added. To this solution was added 2 g of wet cell paste of Providencia sp. PCM 1298. The solution was gently aerated with oxygen gas using a sintered glass sparger over a 24 hour period. The reaction was monitored by periodically removing aliquots and analyzing for the conversion of L-glutamine to 2-oxoglutaramate. The production of 2-oxoglutaramate was monitored by HPLC. After 2 hrs 50% of the L-glutamine was converted to 2-oxoglutaramate, as quantified by HPLC. Conversion of L-glutamine was complete after 24 hours, as detected by HPLC.

I claim:

1. A method for producing 2-oxoglutaramate, comprising the step of incubating bacteria of the genera Providencia or Proteus, or an active biocatalyst derived therefrom, in an incubation solution comprising at least about 25 mM L-glutamine.

2. The method of claim 1, in which the incubation solution comprises at least about 100 mM L-glutamine.

3. The method of claim 1, in which the incubation solution comprises a buffer and catalase and has a pH ranging from 6.5 to 8.5.

4. The method of claim 1, in which the bacteria are one of Providencia sp. PCM-1298 and Providencia sp. PCM-1270.

5. The method of claim 1, in which L-glutamine is added to the incubation solution during incubation over a time period either in two or more aliquots or as a steady trickle.

6. The method of claim 1, further comprising the step of stopping the incubation by either killing the bacteria or removing the bacteria from the incubation solution.

7. The method of claim 1, further comprising the step of purifying the 2-oxoglutaramate by ion exchange chromatography.

8. The method of claim 1, further comprising the step of purifying the 2-oxoglutaramate by precipitation.

9. The method of claim 1, in which the incubation solution is a slurry comprising solid L-glutamine.

10. The method of claim 9, in which the slurry comprises up to about 250 g/L of solid L-glutamine.

11. The method of claim 1 in which the bacteria is Proteus mirabilis.

12. The method of claim 11 in which the bacteria is Proteus mirabilis strain PCM-1353.

13. The method of claim 1 in which the active biocatalyst is immobilized on a substrate.

14. A method for producing 2-oxoglutaramate, comprising the steps of:
a) providing an incubation solution slurry comprising a buffer and solid L-glutamine and having a pH of from about 7.0 to about 8.0;
b) adding to the incubation solution slurry a resuspended wet cell pellet collected from one of a Providencia culture and a Proteus culture; and
c) incubating the slurry to convert L-glutamine to 2-oxoglutaramic acid.

15. The method of claim 14, in which the slurry further comprises catalase.

16. A reaction mixture for producing 2-oxoglutaramate, comprising Providencia or Proteus bacteria or an active biocatalyst derived therefrom and at least about 25 mM L-glutamine.

17. The reaction mixture of claim 16, in which the bacteria are one of Providencia sp. PCM-1298 and Providencia sp. PCM-1270.

18. The reaction mixture of claim 16 in which the bacteria is Proteus mirabilis.

19. The reaction mixture of claim 18 in which the bacteria is Proteus mirabilis strain PCM-1353.

20. The reaction mixture of claim 16, comprising a near-saturation amount of L-glutamine.

21. The reaction mixture of claim 16, comprising a slurry of solid L-glutamine.

22. The reaction mixture of claim 21, in which the slurry comprises up to about 250 g/L solid L-glutamine.

23. The reaction mixture of claim 16, further comprising catalase.

24. The reaction mixture of claim 16, comprising an active biocatalyst derived from Providencia or Proteus bacteria that is immobilized on a substrate.

25. The reaction mixture of claim 16, comprising at least about 1% w/v, wet cell pellet mass, of the bacteria cells.

26. The reaction mixture of claim 21, comprising from 1% by weight to 50% by weight bacteria (wet cell pellet mass), 50 mM TRIS-HCl (pH 7.0 to 8.0), catalase and from 0.32% by weight to 25% by weight L-glutamine.

27. A composition comprising Providencia or Proteus bacteria Land at least about 20 mM 2-oxoglutaramate.

28. The composition of claim 27, in which the bacteria are one of Providencia sp. PCM-1298, and Providencia sp. PCM-1270.

29. The composition of claim 27 in which the bacteria is Proteus mirabilis.

30. The composition of claim 29 in which the bacteria is Proteus mirabilis strain PCM-1353.
